(19) United States
(12) Patent Application Publication
(10) Pub. No.: US 2014/0255485 A1
(43) Pub. Date: Sep. 11, 2014

(54) COLLAGEN HYDROLYSATE AND USE THEREOF

(71) Applicant: GELITA AG, Eberbach (DE)

(72) Inventors: Hans-Ulrich FRECH, Weinheim (DE); Steffen OESSER, Glucksburg (DE); Stephan HAUSMANNS, Heidelberg (DE)

(73) Assignee: GELITA AG, Eberbach (DE)

(21) Appl. No.: 14/282,239

(22) Filed: May 20, 2014

Related U.S. Application Data
(63) Continuation of application No. PCT/EP2012/073180, filed on Nov. 21, 2012.

(30) Foreign Application Priority Data
Nov. 29, 2011 (DE) 10 2011 055 800.4
Mar. 7, 2012 (DE) 10 2012 101 911.8

(51) Int. Cl. A61K 38/39; A61K 45/06

(52) U.S. Cl. CPC A61K 38/39; A61K 45/06
USPC 424/479; 514/17.2; 424/94.4; 424/765; 424/725; 424/729; 424/94.1

(57) ABSTRACT

The present invention relates to collagen hydrolysate and the use thereof for treating and/or preventing cellulite.

### CROSS REFERENCE TO RELATED APPLICATIONS

This patent application is a continuation of International Application Number PCT/EP2012/073180, filed Nov. 21, 2012, which claims the benefit of German applications DE 10 2011 055 800.4, filed Nov. 29, 2011, and DE 10 2012 101 911.8, filed Mar. 7, 2012, which are each incorporated by reference.

### BACKGROUND OF THE INVENTION

The present invention relates to collagen hydrolysate and use thereof for treating and/or preventing cellulite.

Cellulite involves undesirable changes to the properties of the skin and is outwardly noticeable as the formation of depressions in the skin surface. It is almost exclusively women who are affected, although with increasing age, as many as 80% to 98% of women are involved. Cellulite occurs, above all, in areas with significant subcutaneous fat tissue, i.e. on the hips, the buttocks, the abdomen, the upper thighs and the upper arms.

According to the latest research, causes of cellulite are considered to be particular changes in the dermis and the subcutaneous connective tissue, in particular, a contraction of the septa formed from collagen fibres, which connect the reticular dermis to the muscles lying under the subcutaneous fat tissue. This leads overall to a reduction in the elasticity of the skin.

Known treatments for cellulite include, in particular, physical methods such as lymph drainage, ultrasound or vacuum, although these typically produce no, or at least no lasting, success. The topical use of cosmetics such as creams or ointments also does not enable any causative treatment of cellulite because the upper skin layers of the epidermis are not directly involved in the phenomenon.

It is known that through the oral ingestion of collagen hydrolysate, advantageous effects can be achieved with regard to the health of the skin in humans (see V. Zague: “A new view concerning the effects of collagen hydrolysate intake on skin properties” in Arch. Dermatol. Res. 2008 (9) 479). Due to the absorption capability of suitable low molecular weight collagen peptides and the good perfusion of the skin, an accumulation of orally ingested collagen hydrolysate takes place there to a particularly great extent, the concentration being at a maximum in the period from approximately 12 to 24 hours after ingestion (see M. Watanabe Kamiyama et al.: “Absorption and effectiveness of orally administered low molecular weight collagen hydrolysate in rats” in J. Agric. Food Chem. 2010 (58) 835).

Against this background, the present invention proposes a new approach to the causative treatment and/or prevention of cellulite, the approach involving the use of collagen hydrolysate.

Thus, one aspect of the invention concerns collagen hydrolysate as an active ingredient for treating and/or preventing cellulite.

It has also been shown in a clinical trial that, following the administration of collagen hydrolysate, the elasticity of the skin increases measurably. This effect is particularly marked in women aged over 50 years. A greater skin elasticity results in a decreased severity of cellulite.

Furthermore, a variety of in vitro studies have shown that the synthesis of extracellular matrix proteins of the dermal connective tissue is stimulated by collagen hydrolysate. These proteins formed by the skin cells (dermal fribroblasts) comprise collagen (in particular type I), elastin and proteoglycans (such as biglycan, versican and decorin). The synthesis of these proteins in sufficient quantities is decisive for the formation and regeneration of the extracellular matrix of the skin which, in turn, is an essential determining factor for the properties of the dermis, such as elasticity, resilience and moisture regulation.

The collagen hydrolysate used in the context of the invention favourably has a relatively low molecular weight. Preferably, at least 90% by weight of the collagen hydrolysate has a molecular weight of less than 3,500 Da and, more preferably, at least 45% by weight has a molecular weight of less than 1,500 Da. It has been found that more marked effects can be achieved with such particularly low molecular weight components. The molecular weight distribution of the collagen hydrolysate, which is subject to the relevant limit values can be determined very precisely and reproducibly, for example, by means of gel permeation chromatography using a calibration standard made of defined collagen fragments.

The mean molecular weight (mass average molar mass Mw) of the collagen hydrolysate used according to the invention typically lies in the range of approximately 1,700 Da to approximately 2,300 Da.

In a preferred embodiment of the invention, the collagen hydrolysate comprises at least four characteristic peptides with a molecular weight of between 600 Da and 1,200 Da. Collagen hydrolysates contain peptides with different chain lengths or molecular weights which arise when the protein chains of the collagen are split, wherein the molecular weight distributions of these peptides can significantly differ depending on the manufacturing conditions of the hydrolysate. It has surprisingly been found that a collagen hydrolysate with the above-mentioned properties has particularly advantageous effects on the synthesis of matrix proteins, i.e. shows markedly better results than collagen hydrolysates which do not contain the characteristic peptides.

The presence of the characteristic peptides of the collagen hydrolysate can be determined, in particular, by means of MALDI mass spectroscopy in which the characteristic peptides appear as peaks in the mass spectrum. Preferably, the at least four characteristic peptides in a molecular weight distribution found with a mass spectroscopy determined by means of MALDI have an intensity which is at least doubled and, more preferably, at least quadrupled in comparison with their surroundings.

In a preferred embodiment of the invention, the collagen hydrolysate comprises a peptide of between 620 Da and 690 Da, a peptide of between 790 Da and 860 Da, a peptide of between 980 Da and 1,050 Da and a peptide of between 1,175 Da and 1,245 Da. The collagen hydrolysate can also have characteristic peptides of between 1,500 Da and 3,500 Da.

Preferably, the collagen hydrolysate has a hydroxyproline content of 12% by weight or more. The amino acid hydroxyproline formed by the post-translational hydroxylation of proline occurs exclusively in collagen, so that a high proportion of hydroxyproline in the collagen hydrolysate provides a measure of the extensive absence of other connective tissue proteins (e.g. elastin and proteoglycans), fragments of which can also be contained in certain quantities in collagen hydrolysates, depending on the manufacturing methods.

It is favourable if the collagen hydrolysate is manufactured by the enzymatic hydrolysis of gelatine. Gelatine comprises denatured collagen and is obtained by means of various methods known to persons skilled in the art, from the connective tissue or bones of a variety of animal species. In the context of the present invention, the gelatine used as a starting material for collagen hydrolysate is preferably extracted from the skin of mammals, particularly pigs or cattle, although the use of gelatine from poultry is also not excluded. Porcine gelatine, particularly pigskin gelatine is particularly preferred as a starting material.

The enzymatic hydrolysis of the gelatine is typically carried out by means of an endoprotease and it is preferable in the context of the invention to use a plurality of endoproteases (i.e. at least two different endoproteases) in order thereby to influence the amino acid profile of the resulting collagen hydrolysate accordingly, and to increase the positive effect of hydrolysate.

According to a preferred embodiment of the invention, the collagen hydrolysate is produced through the sequential action of at least two endoproteases with different specificity, in particular at least two different metalloproteases and/or serine proteases, that is, proteases which split the amino acid sequence of the collagen molecules either before or after particular amino acids. Favourably, the metalloproteases and/or serine proteases are enzymes from the microorganisms Bacillus subtilis, Bacillus licheniformis, Bacillus amyloliquefaciens, Aspergillus oryzae and Aspergillus melleus.

By means of the selection of suitable endoproteases, not only can the characteristic molecular weight distribution of the collagen hydrolysate be obtained, but the type of amino acids at the termini of the peptides obtained in the hydrolysate is also influenced. In this context, it is preferred, for example, if at least 50% of the N-terminal amino acids of the collagen hydrolysate are hydrophobic amino acids, in particular alanine, leucine and isoleucine.

According to a preferred embodiment of the invention, the collagen hydrolysate is provided for an enteral administration, in particular in the form of oral ingestion. On oral ingestion, more effective transport of the collagen hydrolysate via the blood circulation to the site of action, i.e. in particular to the dermal fibroblasts, is brought about than in the case of a topical administration. Furthermore, this administration form is typically associated with significantly less effort for the user.

Since collagen hydrolysate is extracted from raw materials which are authorised under food laws, it can be used in the context of the present invention, preferably as a nutritional supplement, for treating and/or preventing cellulite. Such nutritional supplements can be identified as “nutraceuticals” or “nutricosmetics”.

The nutritional supplement can be offered in almost any form, for example, as tablets, capsules, sugar-coated pills, pastilles, sachets or a gel or solution (e.g. in single ampoules or in drinks).

Alternatively, the collagen hydrolysate can be contained in a food or luxury food item, for example, in confectionary or in an instant powder for making drinks. The hydrolysate can thus be consumed by the user without additional effort in the context of the normal nutrition (as “functional food”). In this regard, it is particularly advantageous if the collagen hydrolysate is substantially flavourless.

It is favourable if a daily intake of approximately 1.5 g to 5 g, preferably approximately 2 g to 3 g, more preferably approximately 2.3 g to 2.7 g of the collagen hydrolysate is provided. It has been found that through the oral ingestion of this quantity of hydrolysate, a marked effect can be achieved which cannot be substantially enhanced by increasing the daily dose.

When used according to the invention, the collagen hydrolysate can be combined with other active ingredients which have an advantageous effect on the health and particularly on the health of the skin, inter alia with active ingredients having an antioxidant effect. Such active ingredients are preferably selected from vitamins, in particular vitamins C and E, minerals, omega-3 fatty acids, omega-6 fatty acids, omega-9 fatty acids, biotin, lutein, lycopene, caffeine, glucosamine, chondroitin, hyaluronan, folic acid, amino acids, ubiquinone-10, superoxide dismutase and plant extracts from rose hips, lemon verbena or green tea.

in a preferred embodiment of the invention, the administration of the collagen hydrolysate is provided for treating and/or preventing cellulite, particularly in women aged over 50 years and typically women in the postmenopause. In this age group which is generally severely affected by cellulite, the effects are particularly marked, as shown by the clinical trial described below.

The invention also relates to a method for treating and/or preventing cellulite in a patient, the method comprising the administration of collagen hydrolysate to the patient, particularly in the form of oral ingestion.

Preferred embodiments of the method, particularly with regard to the properties of the collagen hydrolysate and the dose to be administered have already been described in relation to the use according to the invention.

The invention also relates to the use of collagen hydrolysate for treating and/or preventing stretch marks in the form of so-called pregnancy stretch marks (Striae gravidarum) which occur particularly in pregnant women. The cause of these stripes is fine tears in the subcutaneous connective tissue caused by severe stretching of the skin. Similarly to cellulite, the occurrence of such tears can also be counteracted by increasing the skin elasticity with the administration of collagen hydrolysate.

A further aspect of the invention concerns the use of collagen hydrolysate for treating and/or preventing local damage to the skin as a result of pressure caused by decubitus ulcers, for example, the occurrence of bedsores. This involves the skin being subject to external pressure, the negative effects of which can be lessened by increasing the elasticity of the skin.

### BRIEF DESCRIPTION OF THE DRAWINGS

This and other advantages of the invention will now be described in greater detail based on the following examples and making reference to the drawings, which show:

FIGS. 1A to 1C: graphical representations concerning stimulation of the synthesis of type I collagen, biglycan or versican;

FIGS. 2A and 2B: graphical representations concerning the increase in skin moisture in hairless mice;

FIG. 3: a graphical representation concerning stimulation of the synthesis of CE proteins;

FIGS. 4A to 4C: MALDI mass spectra of various collagen hydrolysates; and

FIGS. 5A and 5B: graphical representations concerning stimulation of the synthesis of type I collagen, decorin and versican.

### EXAMPLES

1. Production and Properties of the Collagen Hydrolysate

In order to produce a collagen hydrolysate for the use according to the invention, an aqueous solution of a pigskin gelatine (type A, 200 g to 250 g Bloom) at a concentration of between 20% and 40% by weight (of dry material) is used as the starting material. The gelatine is enzymatically hydrolysed by the sequential action of two different endoproteases of microbial origin at 50° C. to 60° C. for between 120 min and 180 min, wherein as the first enzyme, an endoprotease from Bacillus subtilis or from Bacillus amyloliquefaciens is used and as the second enzyme, an endoprotease from Bacillus licheniformis is used. Subsequently, the enzymes are thermally deactivated and the solution is spray dried.

The molecular weight distribution of the resultant collagen hydrolysate can be determined by means of gel permeation chromatography, using the following parameters:
Static phase: TSK 2000 SW XL (Tosoh Bioscience GmbH)
Mobile phase: 0.4 mol/l sodium dihydrogen phosphate pH 5.3
Flow rate: 0.5 ml/min
Calibration standard: defined collagen-type I fragments (FILK, Freiberg)
Detection: UV detector Knauer K-2501 at 214 nm

The determination resulted in the molecular weight distribution for this collagen hydrolysate (hereinafter called low-molecular hydrolysate), as set out in Table 1 below. For comparison purposes, in table 1, the molecular weight distribution of a commercially available collagen hydrolysate determined with the same method (hereinafter called high-molecular hydrolysate) is also shown:

TABLE 1

| Molecular weight | Low-molecular | High-molecular |
|---|---|---|
| range | hydrolysate | hydrolysate |
| >7,500 Da | <5% by weight | <10% by weight |
| 3,500-7,500 Da | ca. 12-18% by weight | ca. 25-35% by weight |
| 1,500-3,500 Da | ca. 25-31% by weight | ca. 29-35% by weight |
| 500-1,500 Da | ca. 40-46% by weight | ca. 24-30% by weight |
| <500 Da | ca. 5-10% by weight | ca. 2-5% by weight |

The hydroxyproline content of this low-molecular hydrolysate is approximately 12% to 13% by weight and, following oxidation with chloramine-T and conversion with p-dimethylaminobenzaldehyde, can be determined photometrically. More than 50% of the N-terminal amino acids of the hydrolysate are hydrophobic amino acids, in particular alanine, leucine and isoleucine.

2. Clinical Trial on the Efficacy of the Collagen Hydrolysate for Cellulite

The efficacy of the low-molecular collagen hydrolysate, produced according to Example 1, for the treatment and/or prevention of cellulite was investigated in a double-blind, randomised placebo-controlled trial. The trial subjects were 69 healthy women aged between 35.3 and 55.4 years, divided into three groups of 23 subjects each. 68 subjects successfully completed the trial.

Beginning six weeks before the start of the trial, no dermatological treatments were permitted to be used and the subjects were also not to change their living and nutritional habits during the trial, nor take any additional nutritional supplements or vitamin preparations or expose their skin to intense UV radiation. No cosmetic preparations were to be used on the volar sides of the forearms, where the effects of the collagen hydrolysate on the skin properties were to be investigated.

Of the three groups, over a period of eight weeks, the first received 2.5 g collagen hydrolysate daily (morning), the second 5 g collagen hydrolysate daily (2.5g each morning and afternoon) and the third received a placebo. For oral ingestion, the hydrolysate could be dissolved in water or a cold drink (with the exception of milk).

Before the first ingestion, after four weeks and after eight weeks, the following parameters of the skin were measured on the volar sides of the left upper arm of the subjects:
Skin elasticity with a Cutometer(k) SEM 575 (mean value from three measurements)
Transepidermal water loss (TEWL) with a DermaLab® device (mean value from three measurements)
Skin moisture content with a Corneometer® CM 825 (mean value from ten measurements)

All measurements were carried out following 30 minutes of acclimatisation in a climate-controlled room at a temperature of 21.5° C. (±1° C.) and a relative air humidity of 50% (±5%).

All three parameters were significantly increased in the groups treated with collagen hydrolysate both after four weeks and after eight weeks. The values measured after eight weeks are given in Table 2 below, specifically as percentage increases, as compared he group given the placebo:

TABLE 2

| Parameter | 2.5 g hydrolysate per day | 5 g hydrolysate per day |
|---|---|---|
| Skin elasticity | ca. 7% | ca. 9% |
| TEWL | ca. 11% | ca. 14% |
| Skin moisture | ca. 6% | ca. 7% |

The increase in skin elasticity shows the effectiveness of the oral administration of collagen hydrolysate for treating and/or preventing cellulite. The improvement in the TEWL and in skin moisture are further advantageous effects of the hydrolysate on skin health and lead, in particular, to an increase in the epidermal barrier function.

An examination of the increase in skin elasticity differentiated by age groups produced the results set out in Table 3, with women under 50 years old (mean age 44.1 years) being compared with women over 50 years old (mean age 53.0 years):

TABLE 3

| Skin elasticity | 2.5 g hydrolysate per day | 5 g hydrolysate per day |
|---|---|---|
| Women under 50 | ca. 3% | ca. 5% |
| Women over 50 | ca. 14% | ca. 15% |

Noticeable here is a particularly marked improvement in the skin elasticity of women aged over 50, who therefore represent a preferred target group for the use according to the invention of collagen hydrolysate.

Skin elasticity was measured again four weeks after completion of the eight-week administration period. Between 92% and 98% of the increases measured after eight weeks were still retained, suggesting a longer lasting effect for the collagen hydrolysate.

3. Stimulation of the Synthesis of Extracellular Matrix Proteins In Vitro

Stimulation of the synthesis of collagen (type I) and of the proteoglycans biglycan and versican was investigated in vitro with human dermal fibroblasts (skin cells). For this purpose, the cells were incubated for 24 hours with 0.5 mg/ml of either the low-molecular or the high-molecular hydrolysate and then the expression of collagen RNA, biglycan RNA and versican RNA was determined by means of real-time PCR and evaluated semi-quantitatively (relative to a control without hydrolysate).

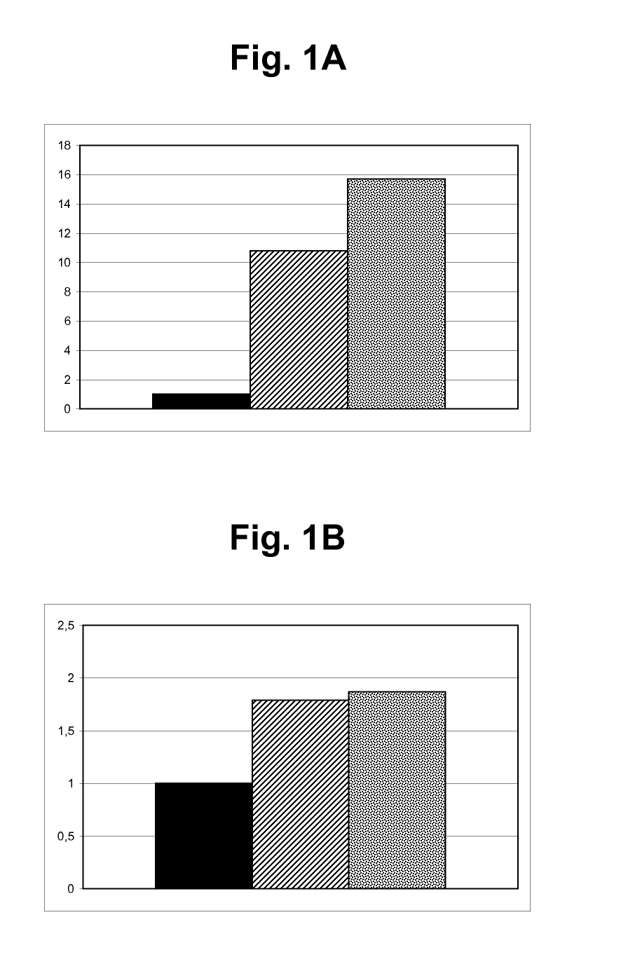

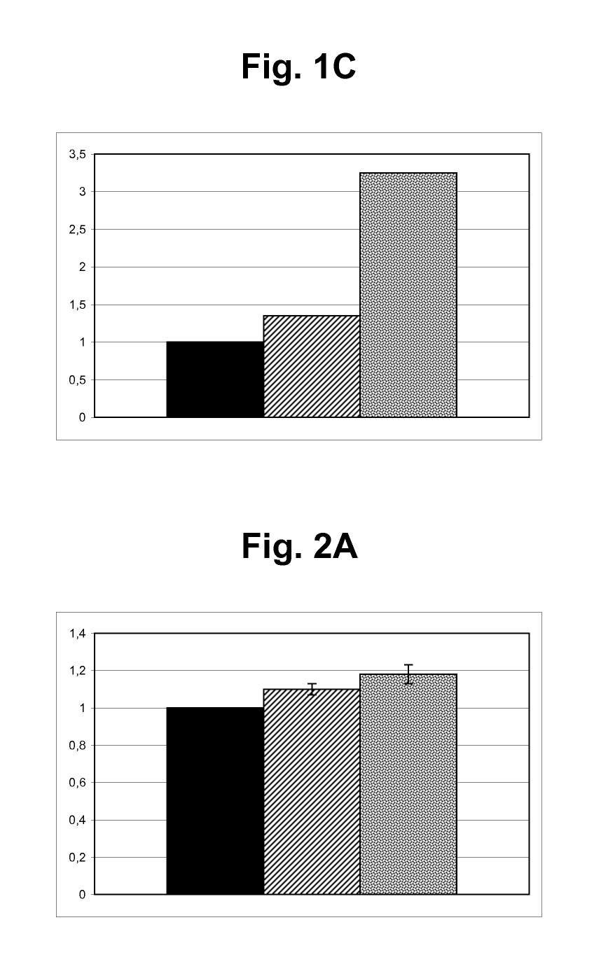

The results are shown as bar charts for type I collagen in FIG. 1A, for biglycan in FIG. 1B and for versican in FIG. 1C, the graphical representations each showing the mean value from at least 18 measurements. Represented on the abscissa is the RNA expression relative to the control (=1). The left-hand solid column, in each case, represents the control, whilst the middle, shaded column is the high-molecular hydrolysate and the right-hand, dotted column is the low-molecular hydrolysate.

It is evident that the synthesis of all three matrix proteins is stimulated by both the collagen hydrolysates, although the positive effect of the low-molecular hydrolysate is more strongly expressed in each case than that of the high-molecular hydrolysate. For collagen which, besides elastin, is mainly responsible for the resilience and elasticity of the skin, and for versican, which plays an important part in the moisture regulation of the skin, the enhanced effect of the low-molecular hydrolysate is particularly clearly expressed.

These stimulating properties of the collagen hydrolysate on the different matrix proteins also offer, apart from the treatment and/or prevention of cellulite according to the invention, a starting point in relation to diseases, for example psoriasis, in which the natural function of the skin is impaired.

4. Increasing the Moisture Content of the Skin in Animal Studies

The influencing of skin moisture with collagen hydrolysate was investigated directly using hairless mice. Hairless mice represent an established model system which is often used for dermatological investigations and the knowledge obtained therefrom can, in principle, be applied to human skin (see e.g. T. Fujimura et al.; 3. Dermatol. Sci. 2000 (24) 105-111 and Y. Nishimori et al.; J. Invest. Dermatol. 2001 (117) 1458-1463).

The animals were fed daily with 150 μg collagen hydrolysate per kg body weight over a period of three weeks, whilst the control group received BSA instead. At the same time, all the animals were given a weekly UV-B radiation dose of 18 mJ/cm² skin surface, by which the skin moisture was negatively influenced.

The moisture content was measured after one week and after three weeks with a Corneometer CM 825 (manufacturer Courage & Khazaka). The measuring principle herein is based on the change in the capacitance of a measuring capacitor due to the dielectric constant of the water bound into the upper skin layers, which differs markedly from the dielectric constant of most of the other substances.

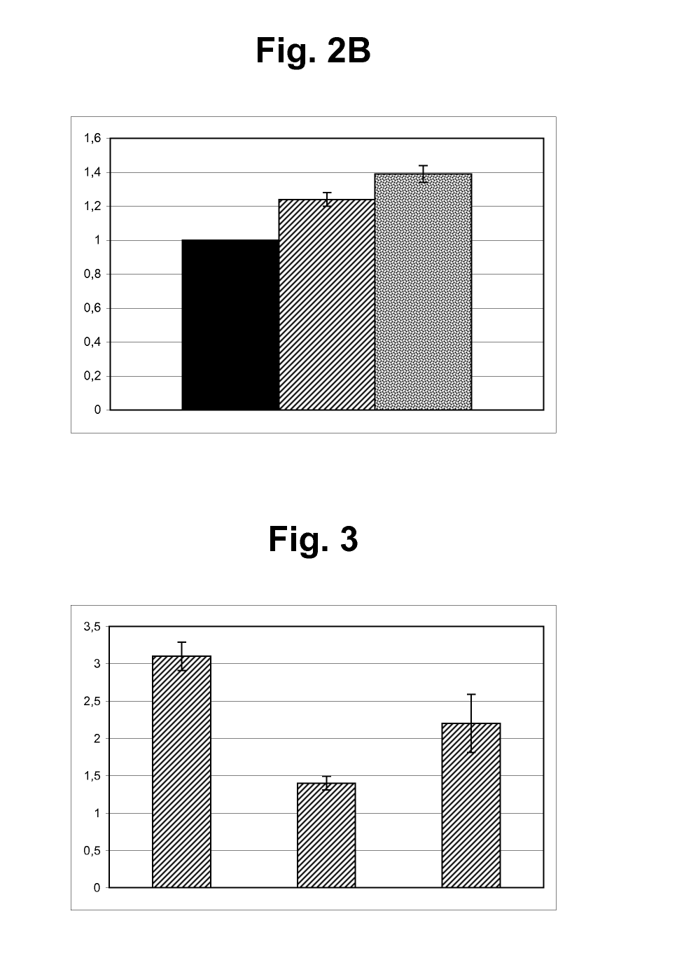

The results are shown as bar charts for the measurement after one week in FIG. 2A and for the measurement after three weeks in FIG. 2B, the graphical representations each showing the mean value and standard error from 7 measurements. Represented on the abscissa is the skin moisture content relative to the control (=1). The left-hand, solid column, in each case, represents the control, the middle, shaded column, the high-molecular hydrolysate and the right-hand, dotted column, the low-molecular hydrolysate.

It is apparent that the increase in skin moisture with the low-molecular hydrolysate is greater both after one week and also after three weeks than with the high-molecular hydrolysate.

5. Stimulation of the Synthesis of CE Proteins In Vitro

So-called “Cornified Envelope” proteins play an important part in the barrier function of the skin against the ingress of pathogenic microbes and toxic substances. The synthesis of the CE proteins involucrin, loricrin and filaggrin was determined in hairless mice which had previously been fed for five weeks with 150 μg collagen hydrolysate per kg body weight daily (as described above). Quantification of the proteins relative to a control group (fed with BSA) was carried out with SDS polyacrylamide gel electrophoresis and Western blot with specific antibodies following extraction of the proteins from the skin.

The results are shown as histograms in FIG. 3, the graphical representation showing the mean value and the standard error from 7 measurements. Represented on the abscissa is the quantity of CE proteins after feeding with the low-molecular hydrolysate relative to the control (=1). The left-hand column represents involucrin, the middle column loricrin and the right-hand column filaggrin.

It is apparent that the synthesis of all three of the CE proteins investigated is stimulated by oral ingestion of collagen hydrolysate and, in the case of involucrin, actually by more than three times.

6. Analysis of the Molecular Weight Distribution Using a MALDI-MS

The low-molecular collagen hydrolysate produced according to Example 1, which has a mean molecular weight of approximately 2,000 Da (hereinafter called hydrolysate A) was compared with two commercially available collagen hydrolysates with a mean molecular weight of approximately 2,100 Da (hereinafter called hydrolysate B) and approximately 2,900 Da (hereinafter called hydrolysate C).

The precise molecular weight distributions of these three hydrolysates were analysed by means of MALDI mass spectroscopy (MALDI-MS). For this purpose, the samples were adjusted to a final concentration of 10 μg/μl in 0.1% trifluoroacetic acid and then purified using μC₁₈ material. The samples were prepared with an HCCA matrix on a MALDI target and the mass spectra were determined using an Ultraflex-III-TOF/TOF mass spectrometer (manufacturer: Bruker Daltonics).

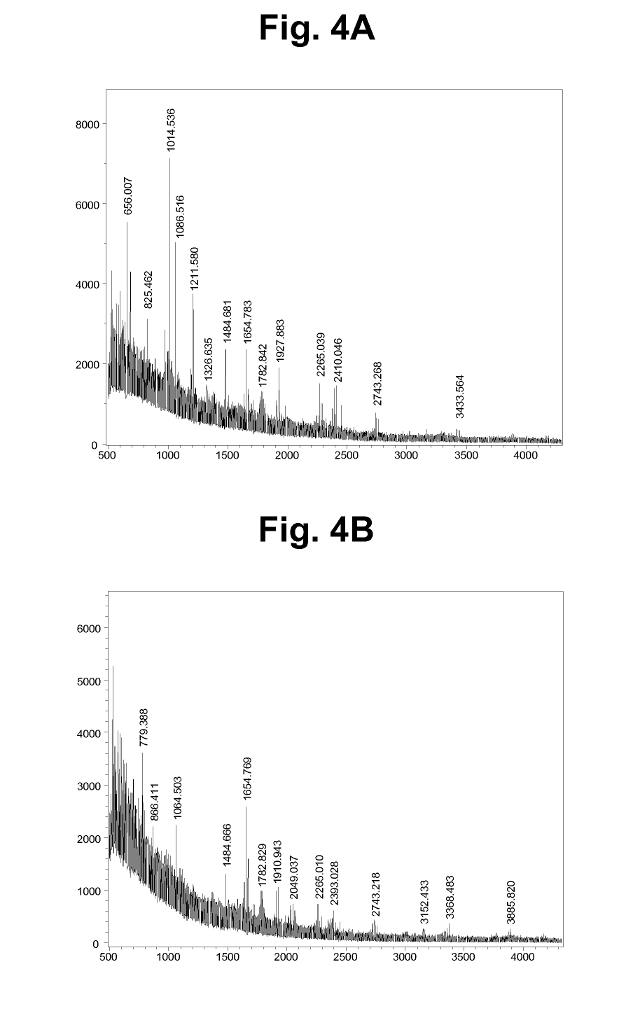

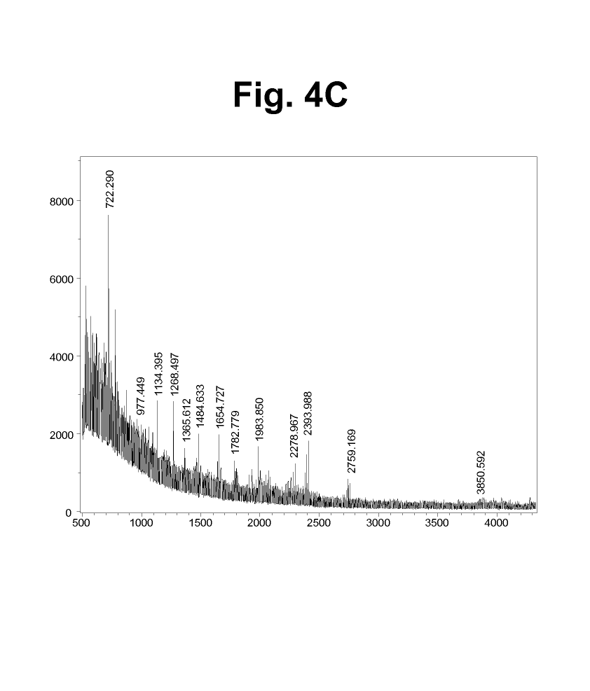

FIGS. 4A to 4C show the corresponding mass spectra or molecular weight distributions of the collagen hydrolysates A, B and C, wherein the molecular weight or mass number are represented on the ordinate and the intensity is represented on the abscissa. A comparison of the three spectra shows that hydrolysate A comprises the following characteristic peptides as per Table 4, the relevant peaks having double to four times the intensity as compared with their surroundings:

TABLE 4

ca. 656 Da
ca. 825 Da
ca. 1,014 Da
ca. 1,211 Da
ca. 1,927 Da
ca. 2,410 Da
ca. 3,433 Da

In particular, the four peptides between 600 Da and 1,500 Da have no correspondences in the two commercial hydrolysates B and C and are therefore particularly characteristic for hydrolysate A.

7. Stimulation of the Synthesis of Extracellular Matrix Proteins In Vitro

Stimulation of the synthesis of collagen (type I) and the proteoglycans decorin and versican was investigated in vitro in human dermal fibroblasts (skin cells). For this purpose, the cells were incubated for 24 hours, with 0.5 mg/ml of each of the hydrolysates A, B and C respectively and then the expression of collagen RNA, decorin RNA and versican RNA was determined by means of real-time PCR and evaluated semi-quantitatively. Decorin plays an important part in the formation of collagen fibres in the skin.

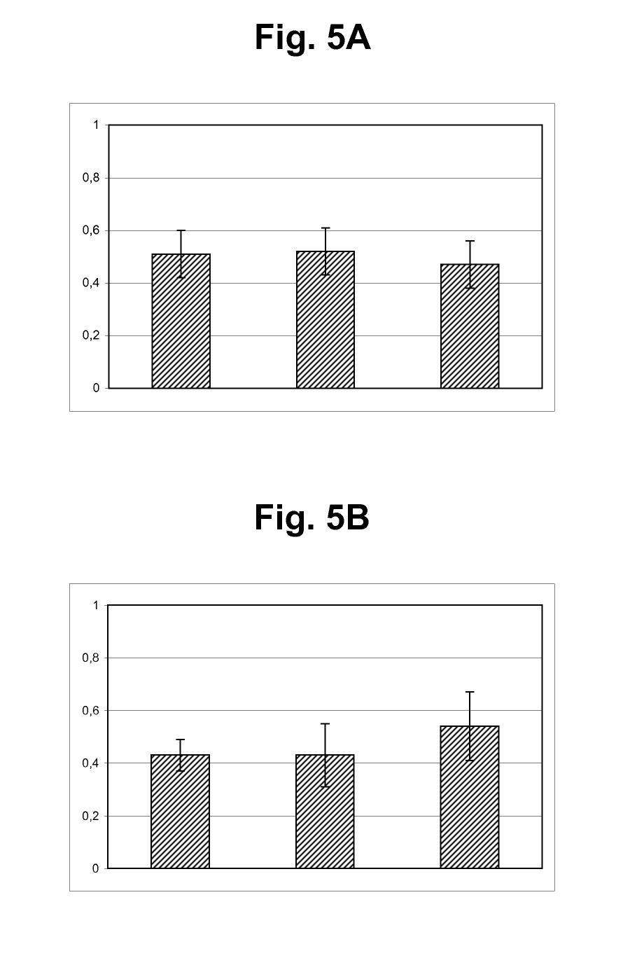

The results are shown as bar charts for hydrolysate B in FIG. 5A and for hydrolysate C in FIG. 5B, the abscissa representing the RNA expression in the commercial hydrolysates B and C, respectively, relative to the RNA expression with hydrolysate A (=1). The left-hand column represents type I collagen, the middle column decorin and the right-hand column versican. In each case, the mean value from at least 7 measurements is shown, together with the standard error.

Interestingly, the data show that, with all three matrix proteins, compared with hydrolysate A, a markedly smaller stimulation of the RNA synthesis takes place with both the hydrolysates B and C, the molecular weights of which are only slightly higher. The characteristic peptides of hydrolysate A therefore appear to play a decisive role in the advantageous effect thereof.

8. Example Recipes for Nutritional (Supplement) Product

Some example recipes for the use according to the invention of the collagen hydrolysate are given below, although these can naturally be amended in many ways:

Capsettes (Nutritional supplement)

| | | |
|---|---|---|
| Glycerine | 53.67% | by weight |
| Collagen hydrolysate | 21.95% | by weight |
| Gelatine | 10.08% | by weight |
| Guar gum | 6.00% | by weight |
| Lecithin | 5.00% | by weight |
| Citric acid | 2.00% | by weight |
| Flavouring (cassis) | 0.50% | by weight |
| Orange oil | 0.50% | by weight |
| Acesulfame K | 0.30% | by weight |

Chocolate

| | |
|---|---|
| Cocoa mass | 51.0% by weight |
| Sucrose | 22.4% by weight |
| Cocoa butter | 16.6% by weight |
| Collagen hydrolysate | 10.0% by weight |

Drink

| | | |
|---|---|---|
| Water | 63.00% | by weight |
| Aloe vera concentrate | 31.00% | by weight |
| Collagen hydrolysate | 4.00% | by weight |
| Sucrose | 1.50% | by weight |
| Citric acid | 0.26% | by weight |
| Flavourings and | 0.24% | by weight |
| colouring agents | | |
| Sucralose | 0.0031% | by weight |

1. A method for treating and/or preventing cellulite in a patient, the method comprising administering collagen hydrolysate to the patient.

2. The method according to claim 1, wherein at least 90% by weight of the collagen hydrolysate has a molecular weight of less than 3,500 Da and at least 45% by weight of the collagen hydrolysate has a molecular weight of less than 1,500 Da.

3. The method according to claim 1, comprising administering collagen hydrolysate comprising at least four peptides with a molecular weight of between 600 Da and 1,200 Da.

4. The method according to claim 3, wherein the at least four peptides in a molecular weight distribution found by means of MALDI mass spectroscopy have an intensity which is at least doubled in comparison with their surroundings.

5. The method according to claim 1, comprising administering collagen hydrolysate comprising a peptide of between 620 Da and 690 Da, a peptide of between 790 Da and 860 Da, a peptide of between 980 Da and 1,050 Da and a peptide of between 1,175 Da and 1,245 Da.

6. The method according to claim 1, wherein the collagen hydrolysate further comprises peptides with a molecular weight of between 1,500 Da and 3,500 Da.

7. The method according to claim 1, comprising administering collagen hydrolysate having a hydroxyproline content of 12% by weight or more.

8. The method according to claim 1, wherein the collagen hydrolysate is manufactured by the enzymatic hydrolysis of gelatine.

9. The method according to claim 8, the gelatine being a porcine gelatine.

10. The method according to claim 8, wherein the collagen hydrolysate is produced through the sequential action of at least two endoproteases with different specificity.

11. The method according to claim 10, wherein the endoproteases are metalloproteases and/or serine proteases selected from enzymes from the microorganisms Bacillus subtilis, Bacillus licheniformis, Bacillus amyloliquefaciens, Aspergillus oryzae and Aspergillus melleus.

12. The method according to claim 1, wherein at least 50% of N-terminal amino acids of the collagen hydrolysate are hydrophobic amino acids.

13. The method according to claim 1, comprising administering the collagen hydrolysate to the patient orally.

14. The method according to claim 13, wherein the collagen hydrolysate is a nutritional supplement in the form of tablets, capsules, sugar-coated pills, pastilles, sachets, a gel or a solution.

15. The method according to claim 13, wherein a daily intake of approximately 1.5 g to 5 g of the collagen hydrolysate is adminstered to the patient.

16. The method according to claim 1, comprising administering the collagen hydrolysate in combination with one or more further active ingredients which are selected from vitamins, minerals, omega-3 fatty acids, omega-6 fatty acids, omega-9 fatty acids, biotin, lutein, lycopene, caffeine, glucosamine, chondroitin, hyaluronan, folic acid, amino acids, ubiquinone-10, superoxide dismutase and plant extracts from rose hips, lemon verbena or green tea.

17. The method according to claim 1, wherein the patient is a woman aged over 50 years.

18. (canceled)

19. The method according to claim 12, wherein at least 50% of the N-terminal amino acids of the collagen hydrolysate are alanine, leucine and isoleucine.

20. The method according to claim 15, wherein a daily intake of approximately 2 g to 3 g of the collagen hydrolysate is administered to the patient.

21. The method according to claim 20, wherein a daily intake of approximately 2.3 g to 2.7 g of the collagen hydrolysate is administered to the patient.

22. The method according to claim 17, wherein the patient is a postmenopausal woman.
